(19) United States
(12) Plant Patent Application Publication
(10) Pub. No.: US 2005/0216984 P1
(43) Pub. Date: Sep. 29, 2005

(54) FICUS PLANT NAMED 'MARGARITA'

(50) FICUS BENJAMINA; MARGARITA

(75) Inventor: Jan van Geest, s'Gravenzande (NL)

Correspondence Address: FOLEY AND LARDNER;SUITE 500, 3000 K STREET NW, WASHINGTON, DC, 20007, US

(73) Assignee: Plantenkwekerij J. van Geest, B.V.

(21) Appl. No.: 10/806,238

(22) Filed: Mar. 23, 2004

(51) Int. Cl. A01H 5/00

(52) U.S. Cl. PLT/211

(57) ABSTRACT

A new and distinct cultivar of Ficus plant named ‘Margarita’, as described and illustrated, and particularly characterized by the variegated yellow-green (lime colored) foliage with a dark green amorphous, irregularly shaped and irregularly patterned very dark green spot, located along the midrib of the leaf; and by a heavily branching, upright growth habit.

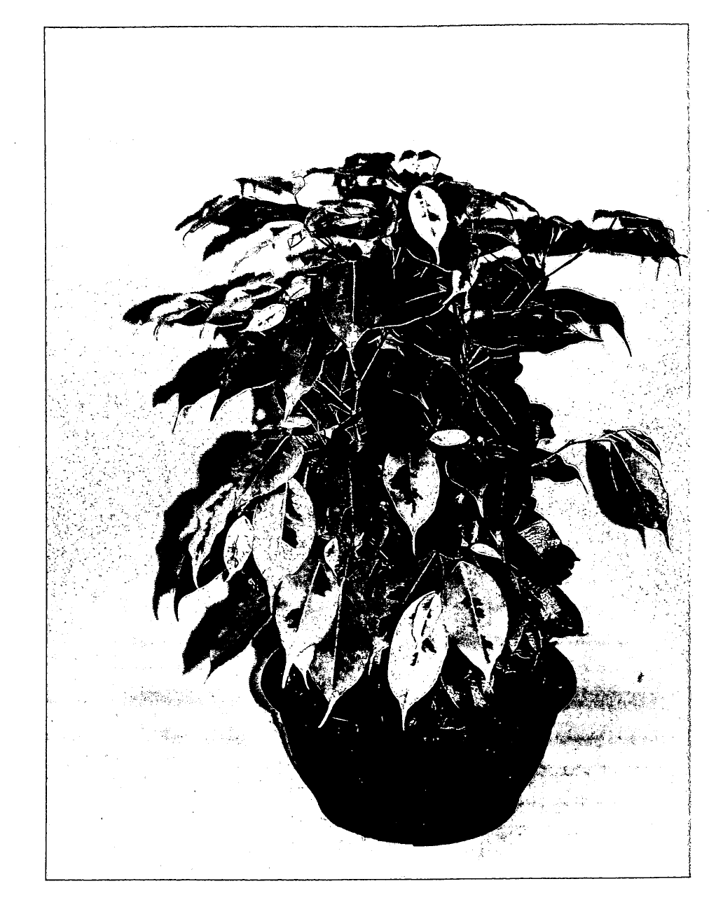

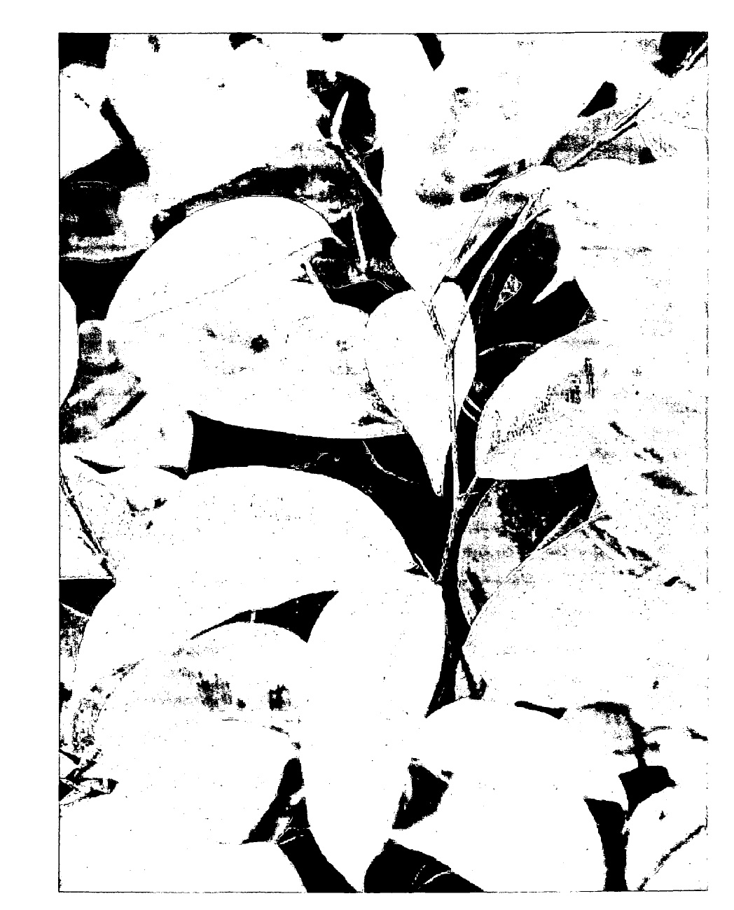

Latin name of the genus and species of the plant claimed:

Ficus benjamina

Variety denomination:

Margarita

### BACKGROUND OF THE INVENTION

The present invention relates to a new and distinct cultivar of Ficus benjamina. The varietal denomination of the new cultivar is ‘Margarita’. The new variety was discovered as a mutation in a controlled planting of Ficus benjamina ‘Midnight’ in a greenhouse in s'Gravenzande, The Netherlands in March, 2001.

### SUMMARY OF THE INVENTION

The new variety is a mutation of the Ficus variety ‘Midnight’ (U.S. Plant Pat. No. 9,856), and was selected by the inventor, Jan van Geest. The mutation was selected among plants of ‘Midnight’ which were grown in a controlled environment in a glasshouse in s'Gravenzande, The Netherlands in March, 2001. The new variety was first asexually propagated by vegetative cutting in April 2001, in s'Gravenzande, The Netherlands. Asexual reproduction through succeeding generations has established that the combination of characteristics as herein disclosed for the new cultivar are firmly fixed and retained through successive generations of asexual reproduction.

In comparison to the parent plant, ‘Midnight’, the leaves of ‘Margarita’ are variegated, having lime leaf green (near RHS 145A, new foliage, upper side) with dark green amorphous, irregularly shaped and irregularly patterned, very dark green (RHS 139B) spot located along the midrib of the leaf. The leaves of ‘Midnight’ by comparison are 137B (new foliage, upper side). In addition, ‘Margarita’ varies from the parent plant in leaf and petiole size. The leaves of ‘Margarita’ are narrower, 3.5 cm, as compared to 4 cm leaf width of ‘Midnight’, and shorter, 9 cm, as compared to 11 cm long leaves of ‘Midnight’. The petioles of ‘Margarita’ are shorter, approximately 10 mm in length, as compared to 18 mm long petioles of ‘Midnight’.

### BRIEF DESCRIPTION OF THE INVENTION

The following traits have been repeatedly observed and are determined to be basic characteristics of ‘Margarita’ which in combination distinguish this Ficus as a new and distinct cultivar from parent cultivar ‘Midnight’ and other Ficus cultivars:

1. Variegated colored leaves, having a lime colored leaf with a dark green spot;

2. irregularly shaped leaves; and

3. irregular patterned spots of very dark green located along the midrib of the leaf

4. heavily branching, upright growth habit.

### BRIEF DESCRIPTION OF THE DRAWINGS

The accompanying photographic drawing illustrates the overall appearance of the new Ficus cultivar, showing colors as true as is reasonably possible with color reproductions of this type. The first photographic drawing shows the typical characteristics of Ficus ‘Margarita’ in its entirety. The second photographic drawing is a close up of the foliage of ‘Margarita’.

### DETAILED DESCRIPTION OF THE NEW VARIETY

‘Margarita’ has not been observed under all possible environmental conditions. The phenotypic expression may vary with variations in environment such as temperature, light intensity, day length and cultural conditions, without a change in the genotype of the plant.

The following observations and descriptions are of a 6 month old plant grown in Homestead, Fla. in an unheated, open air shade house from January, 2003 until July, 2003. The shade cloth on the structure has a 73% shade factor. In this description, color references are to The Royal Horticultural Society Colour Chart (RHS). The color references are approximate as color depends to a degree on horticultural practices such as light level and fertilization rates.

### PLANT

Form: Medium compact, upwardly branching, ascending regularly and richly branching, somewhat weeping.
Growth: Lateral branches are approximately at a 30 to 45 degree angle to the trunk, but slightly weeping at top.
Growth habit: Heavily branching, upright.
Height attained: Vigorous, about 1 meter.
Foliage:
   Size.—About 3.5 cm wide by about 9 cm long (to leaf tip).
   Quantity.—Multiple, numerous.
   Color.—New foliage: upper side: yellow-green, near RHS 145 A with a green spot along the midrib, near RHS 139 B; underside: yellow-green, near RHS 145 A, with a green spot along the midrib, near RHS 139 C. Old foliage: upper side: yellow-green, near RHS 144 A, with a green spot along the midrib, near RHS 139 A; underside: yellow-green, near RHS 144 B, with a green spot along the midrib near RHS 137 C.
   Shape.—Ovate with acuminate leaf tip, slightly coarsely sinuate and glabrous.
   Texture.—Smooth, leathery, glossy; underside smooth, matte.
   Veination.—Single mid-rib vein running from petiole to leaf tip, convex on the underside, light green, near RHS 145 B.
   Edge.—Smooth, slightly undulated.
   Serration.—None.
   Stipules: Leaves are glabrous.
   Auricle.—Absent.
   Petiole.—Near RHS 144 A; about 10 mm long.
   Resistance to disease.—No particular observations made.
   Rachis.—None.
Wood:
   New shoots.—Color: near RHS 144 A; smooth.
   Old wood.—Color near RHS 197 A (no flowers or fruit observed).
   Temperature range.—To 2° C.
   Internode spacing.—About 25 mm.
   Preferred growing conditions.—20° C. to 32° C.
Reproductive organs: None observed.

1. A new and distinct variety of Ficus plant designated ‘Margarita’, substantially as illustrated and described herein.
